(19) United States
(12) Patent Application Publication
(10) Pub. No.: US 2023/0313246 A1
(43) Pub. Date: Oct. 5, 2023

(54) Application of Strain CH18 in Preparation of Anthocyanins and Other Flavonoid Compounds

(71) Applicant: Beijing Institute of Technology, Beijing (CN)

(72) Inventors: Yanju Li, Beijing (CN); Zhengli Yang, Beijing (CN); Yongni Wu, Beijing (CN); Aiping Zhang, Beijing (CN)

(21) Appl. No.: 18/132,514

(22) Filed: Apr. 10, 2023

(51) Int. Cl. C12P 17/06; C12N 1/14

(52) U.S. Cl. CPC C12P 17/06; C12N 1/145; C12R 2001/77

(57) ABSTRACT

The present invention discloses application of a strain CH18 in preparation of anthocyanins and other flavonoid compounds. The preservation number of the strain CH18 is CGMCC NO.14784. Flavonoid compounds have the functions of resisting oxidation, tumors and fatigue, delaying senescence, preventing cardiovascular and cerebrovascular diseases, reducing blood pressure and blood lipid, regulating neuroendocrine, resisting osteoporosis, removing freckles, resisting wrinkles, and improving eyesight. The present invention uses microorganisms to produce anthocyanins and other flavonoids, which has the advantages of low price, easy control, short production cycle and year-round production, and can meet the needs of industrial production. Therefore, the present invention opens up a new biological pathway for producing a variety of anthocyanins and other flavonoid compounds with high efficiency and low cost.

### TECHNICAL FIELD

The present invention belongs to the technical field of development and utilization of microbial resources, and particularly relates to application of a strain CH18 in preparation of anthocyanins and other flavonoid compounds.

### BACKGROUND

Anthocyanin is an important flavonoid compound that is one of the main pigments that make up the color of flowers and fruits. According to different structures, the common anthocyanins in plants can be divided into six categories: cyanindin, pelargonidin, delphinidin, peonidin, petunidin and malvidin. Flavonoid compounds refer to a series of polyphenol compounds with 2-phenylchromone as the parent nucleus, which belong to plant secondary metabolites. In addition to anthocyanins, flavonoids comprise different substances such as flavonol, isoflavone, flavonoid, flavanone, chalcone and dihydroflavone (dihydroflavonol). Flavonoids in plants are mostly in the form of glycosides, and flavone glycosides form various flavonoid glycoside substances due to different factors such as hydroxyl number, number of methylation, acetylation or glycosylation, type of sugar and joint position. More than 5000 flavonoid compounds have been identified in edible plant foods. Flavonoids derived from different plants have different components.

Flavonoid compounds have various biological activities and pharmacological activities such as resisting fatigue, bacteria, virus, tumors, oxygen free radicals and inflammation, relieving pain, protecting liver, preventing cardiovascular and cerebrovascular diseases, reducing blood pressure and blood lipid, regulating neuroendocrine, resisting osteoporosis, removing freckles, resisting wrinkles, improving eyesight, enhancing body immunity, relieving cough, eliminating phlegm and relieving spasm.

At present, flavonoid compounds such as anthocyanins are mainly derived from plants. Plants have long production cycle, and are affected by multiple factors such as environment, place of origin and seasonal supply of raw materials. Compared with that of plants, the use of microorganisms to produce flavonoids such as anthocyanins has the advantages of short production cycle, low consumption, high efficiency and year-round production, and can meet the needs of industrial production, with a broad application prospect. The strain obtained by screening in the present invention can be synthesized into a variety of anthocyanins and other flavonoid compounds, which opens up a new biological pathway for the production of flavonoid compounds such as anthocyanins.

### SUMMARY

The present invention discloses application of a strain CH18 in preparation of anthocyanins and other flavonoid compounds, and the strain CH18 is Fusarium oxysporum. The strain is preserved at China General Microbiological Culture Collection Center (CGMCC) on Oct. 11, 2017, the preservation address is: Building 3, Courtyard 1, Beichen West Road, Chaoyang District, Beijing, and the preservation number is CGMCC NO.14784.

The present invention also discloses application of a strain CH18 in preparation of products of anthocyanins and other flavonoid compounds, and the preservation number of the strain CH18 is CGMCC NO.14784.

The present invention also discloses application of a strain CH18 in preparation of products for resisting bacteria and virus, increasing leucocytes, resisting tumors, reducing blood pressure, blood lipid and natural pigment, scavenging free radicals or exciting the central nervous system, wherein the preservation number of the strain CH18 is CGMCC NO.14784; and bioactive substances of the products are anthocyanins and other flavonoid compounds;

The anthocyanins are anthocyanins or proanthocyanidins;

Preferably, the proanthocyanidins comprise proanthocyanidins A and proanthocyanidins B; preferably, the proanthocyanidins A are proanthocyanidins A2; and preferably, the proanthocyanidins B are proanthocyanidins B2;

Preferably, the anthocyanins comprise one or more of cyanindin, pelargonidin, delphinidin, peonidin, petunidin (Pt) and malvidin.

Preferably, the cyanindin comprises one or more of cyanidin, cyanidin glucoside and cyanirin-3-O-arabinoside;

Preferably, the pelargonidin comprises one or more of pelargonin, pelargonin 3-sophorose 5-glucoside, pelargonin-3,5-glucoside and pelargonin-3-glucoside;

Preferably, the delphinidin comprises one or more of delphinidin glucoside and delphinidin-3-O-arabinoside;

Preferably, the peonidin comprises one or more of peonidin 3-rhamnose-5-glucoside, peonidin glucoside and peonidin-3-O-arabinoside;

Preferably, the petunidin comprises petunidin glucoside or petunidin-3-O-arabinoside;

Preferably, the malvidin refers to malvidin-3-O-glucoside.

The basic structural unit of anthocyanin in the application is 3,5,7-hydroxy-2-phenyl-benzopyran;

Preferably, the other flavonoid compounds are one or more of flavonol, isoflavone, flavanone, flavonoid, chalcone and dihydroflavone (dihydroflavonol);

Preferably, the flavonol refers to one or more of quercetin, rutin, myricetrin, kaempferide, troxerutin, icariin, hyperoside, fisetin, isorhamnetin, astragalin, myricetin, kaempferol and quercitrin.

Preferably, the isoflavone refers to one or more of puerarin, genistein, morin, daidzin, genistin, biochanin A, glycitin and glycitein;

Preferably, the flavanone (flavanonol) refers to one or more of (+)-catechin, epigallocatechin gallate and epicatechin;

Preferably, the chalcone refers to one to more of phloretin and isoliquiritigenin;

Preferably, the dihydroflavone (dihydroflavonol) refers to one or more of silybin, hesperidin, distylin, neohesperidin, astilbin, naringenin, hesperetin, dihydroquercetin, dihydrokaempferol and dihydromyricetin;

Preferably, the flavonoid refers to one or more of apigenin, baicalein, baicalin, luteolin, diosmin, chrysin, vitexin, acacetin and tangeretin;

The products are foods, health care products, medicines, cosmetics and agricultural or daily chemical products;

Preferably, the foods are dairy products, meat products, wine processed products, alcoholic beverages, acid beverage, flour product or pickles;

Preferably, the health care products, medicines, cosmetics or natural pigments are bacteria powder, tablets, capsules, paste or liquid;

Preferably, the agricultural or daily chemical products are pH test liquid, test powder, test paper and additives;

The key intermediate products synthesized from anthocyanins such as naringenin, dihydrokaempferol, dihydromyricetin, dihydroquercetin, cyanidin and pelargonin detected from the targeting metabolome reveal three pathways for synthesizing anthocyanins from strain CH18: a dihydrokaempferol (DHK) pathway, a dihydroquercetin (DHQ) pathway and a dihydromyricetin (DHM) pathway, which can be used to regulate and simulate the production of anthocyanin compounds by fermentation of the strain CH18.

The present invention discloses application of a strain CH18 or fermentation liquor thereof in preparation of flavonoid compounds, and the flavonoid components comprise multiple anthocyanin components and 45 other flavonoid components. The preservation number of the strain CH18 is CGMCC NO.14784.

The present invention has the following beneficial effects: the strain CH18 can be synthesized into multiple anthocyanins and 45 other flavonoid components simultaneously, and the synthesized flavonoids have high yield, which provides a new resource and a new pathway for microbial production of anthocyanins, other flavonoid compound products and various purified products.

The existing flavonoid compounds are mainly obtained from plants, the supply of plant raw materials is seasonally limited, and cultivation requires a large amount of farmland. The production of flavonoid compounds from the strain CH18 has the advantages of low price, easy control, short production cycle and year-round and fermentation and production, and can meet the needs of industrial production. Therefore, the present invention opens up a new pathway for producing flavonoid compounds with high efficiency and low cost. The anthocyanins from the strain can be used in the fields of health care, foods, cosmetics, hair care and stabilization, environmental water quality testing, industry and agriculture, with a broad application prospect.

### DESCRIPTION OF DRAWINGS

FIG. 1 is a total ion current (TIC) of targeted determination of a synthetic product of a strain CH18.

FIG. 2 is a chromatogram of delphinium-glucoside synthesized from a strain CH18 for LC-MS/MS targeted detection; the left figure is a control article, and the right figure is a sample of strain CH18.

FIG. 3 is a total ion current of LC-MS/MS determination of a synthetic product of a strain CH18.

FIG. 4 is a mass spectrogram of peonidin 3-rhamnose 5-glucoside synthesized from a strain CH18 for LC-MS/MS detection.

FIG. 5 shows color changes of fermentation liquor of a strain CH18 at different pH values, with the pH values of 2, 3, 4, 5, 6, 7, 8, 9, 10 and 11 from left to right.

FIG. 6 shows light stability test results of anthocyanins from a strain CH18.

FIG. 7 shows the impacts of metal ions on the stability of anthocyanin extract of a strain CH18.

FIG. 8-FIG. 16 are MRM chromatograms of 49 flavonoids in standards and metabolic samples of a strain CH18.

### DETAILED DESCRIPTION

The technical solution in the embodiment of the present invention will be clearly and fully described below. Apparently, the described embodiment is merely part of the embodiments of the present invention, not all of the embodiments. Based on the embodiments in the present invention, all other embodiments obtained by those ordinary skilled in the art without contributing creative labor will belong to the protection scope of the present invention.

The strain CH18 is preserved at China General Microbiological Culture Collection Center (CGMCC) on Oct. 11, 2017, the preservation number is CGMCC NO.14784, and the preservation address is: Institute of Microbiology, Chinese Academy of Sciences, Building 3, Courtyard 1, Beichen West Road, Chaoyang District, Beijing.

Embodiment 1 Fermentation culture of strain CH18

A strain CH18 is inoculated in an inorganic liquid medium or PDA medium, cultured in a shaker with the temperature of 22-30° C. and the revolving speed of 160 r/min-200 r/min for 5-20 d, sampled at different times, extracted with n-butanol solution, and filtered with a 0.22 um or 0.45 um filter membrane to obtain a CH18 sample to be tested, for measurement of anthocyanins and other flavonoid components.

The components of the medium used are as follows:

PDA medium: 200 g of potato, 10-30 g of glucose, 1 L of distilled water with a natural pH value, sterilized at 115° C. for 20-30 min. 10-15 g of agar is added to solid medium.

Preparation of inorganic medium: 0.5 g of dipotassium phosphate, 0.5 g of monobasic potassium phosphate, 0.2 g of sodium chloride, 2.0 g of ammonium nitrate, 0.2 g of magnesium sulfate, 0.1 g of calcium chloride, 0.01 g of manganese sulfate, 0.01 g of ferrous sulfate, 10-30 g of glucose and 1000 mL of distilled water, shaken up, and sterilized at 115° C. for 20-30 min.

Embodiment 2 Detection of flavonoids synthesized from strain CH18 based on HPLC-MS/MS targeted metabolome

1. Materials and Methods

(1) Reagents

Methanol and acetonitrile are chromatographic reagents purchased from Sigma Corporation. Formic acid is purchased from Shanghai Aladdin Biochemical Technology Co., Ltd.

Standards: naringenin, dihydrokaempferol, dihydromyricetin, dihydroquercetin, pelargonin chloride, cyanidin, cyanidin glucoside, delphinidin glucoside and anthocyanin B2, purchased from Beijing Solarbio Science & Technology Co., Ltd., and others purchased from Shanghai Yuanye Bio-Technology Co., Ltd.

(2) Instruments

TGL-16 high speed freezing centrifuge (Hunan Xiangyi) and vortex oscillator (Hunan Xiangyi),

Liquid chromatograph Waters Acquity UPLC and mass spectrograph AB SCIEX 5500 QQQ-MS,

Chromatographic column: Acquity UPLC HSS T3 (1.8 μm, 2.1 mm*100 mm).

(3) Sample Treatment

Putting 500 μL of sample in a 2 mL centrifuge tube, and freeze-drying. Then adding the freeze-dried sample to 0.400 mL of 80% methanol solution (containing 200 ng/mL internal standard), fully shaking for 1 min, and conducting ultrasonic treatment at 4° C. for 30 min; and standing at 4° C. for 60 min, centrifuging for 10 min, and collecting supernatant for detection.

(4) UPLC-QQQ-MS Method

Chromatographic separation condition: column temperature: 40° C.; mobile phase composition: A—water (0.1% formic acid), and B—acetonitrile; flow rate: 0.300 mL/min. Running time: 18 min, and sample size: 6 μL. The gradient elution program of the sample is shown in Table 1.

TABLE 1
Gradient Elution Program of Sample

| Time | % B |
|---|---|
| Initial | 10.0 |
| 1.00 | 10.0 |
| 14.00 | 90.0 |
| 15.00 | 90.0 |
| 15.10 | 10.0 |
| 18.00 | 10.0 |

(5) Mass Spectrometry Conditions

Electrospray ion source (ESI); multiple reaction monitoring (MRM); curtain gas: 35 arb; collision gas: 7 arb; ion spray voltage: ±4500 V; ion source temperature: 450° C.; ion source gas 1: 55 arb: and ion source gas 2: 55 arb.

(6) MRM data Collection

Adding the prepared standard solution into a sample bottle for sampling. Collecting data by the MRM method. Conducting mass spectrometry MRM of flavonoid products in the fermentation sample of the strain CH18.

2. Detection Results of Targeted Metabolome of Flavonoids Synthesized from Strain CH18

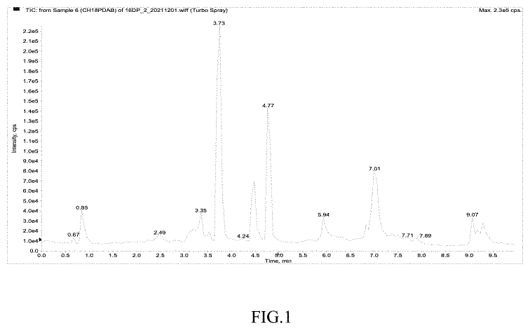

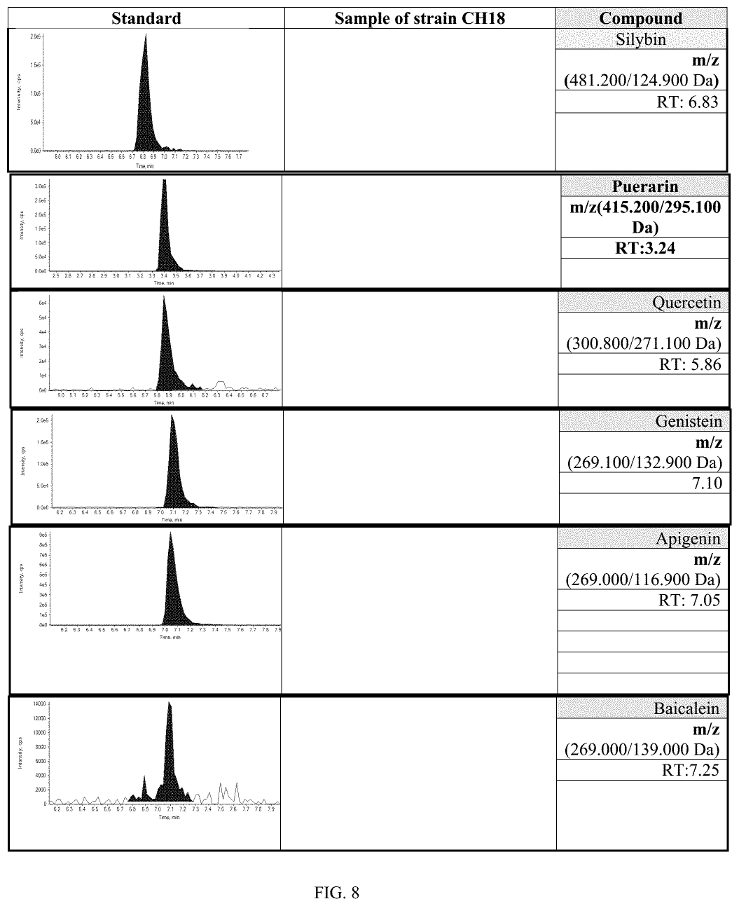

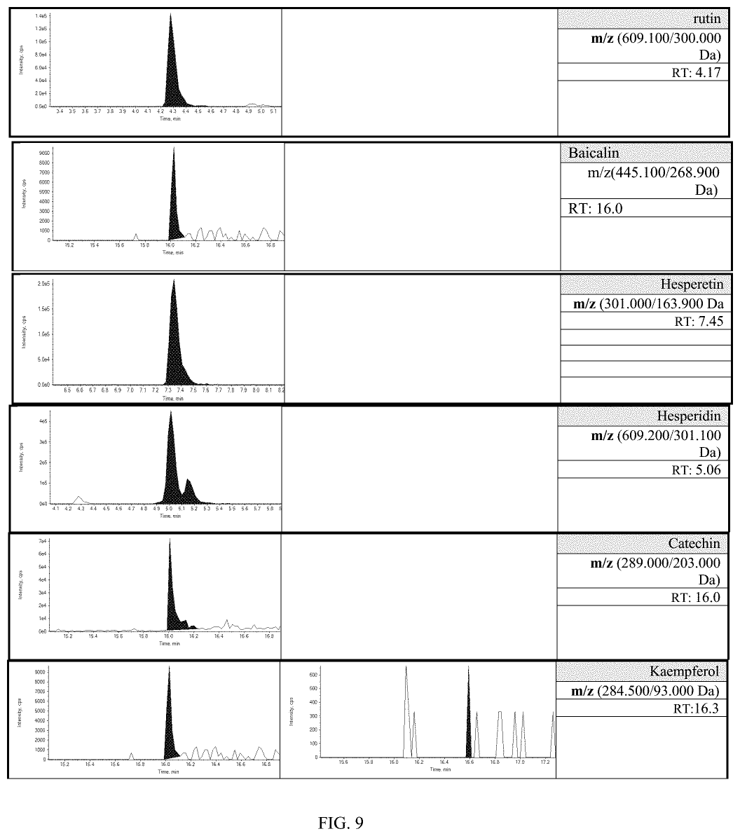

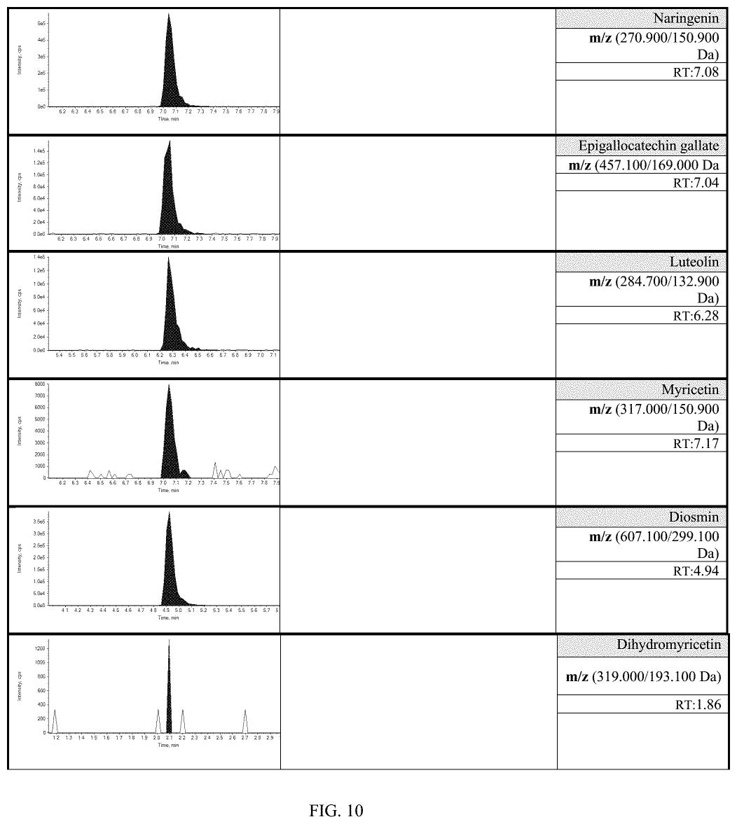

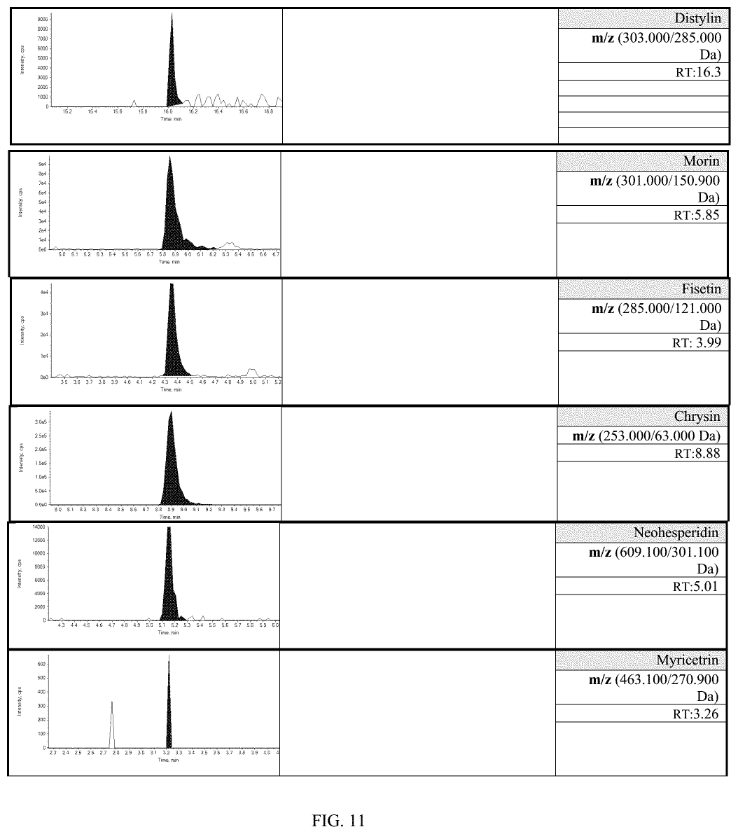

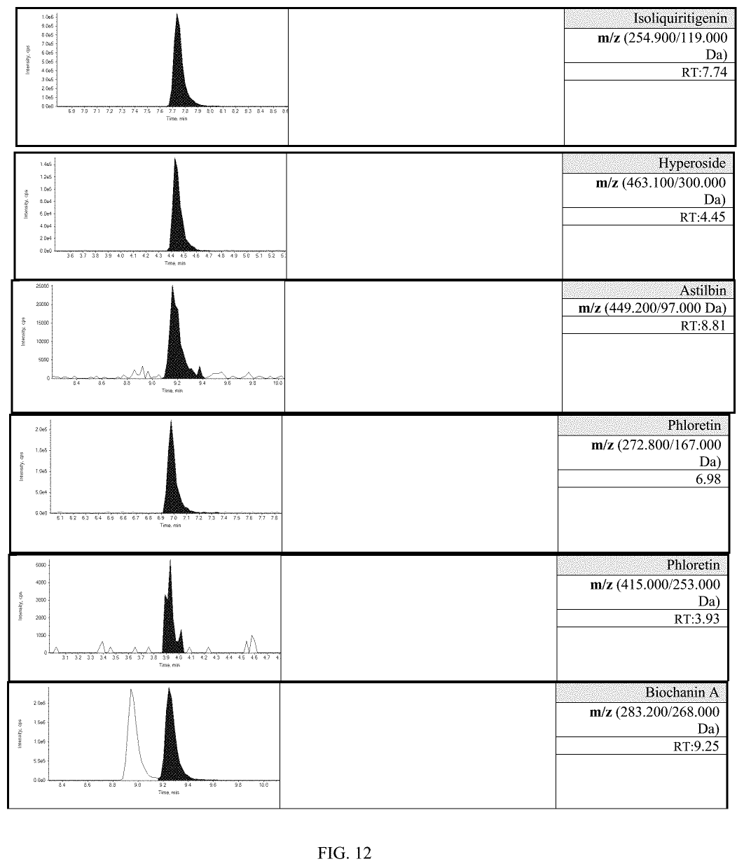

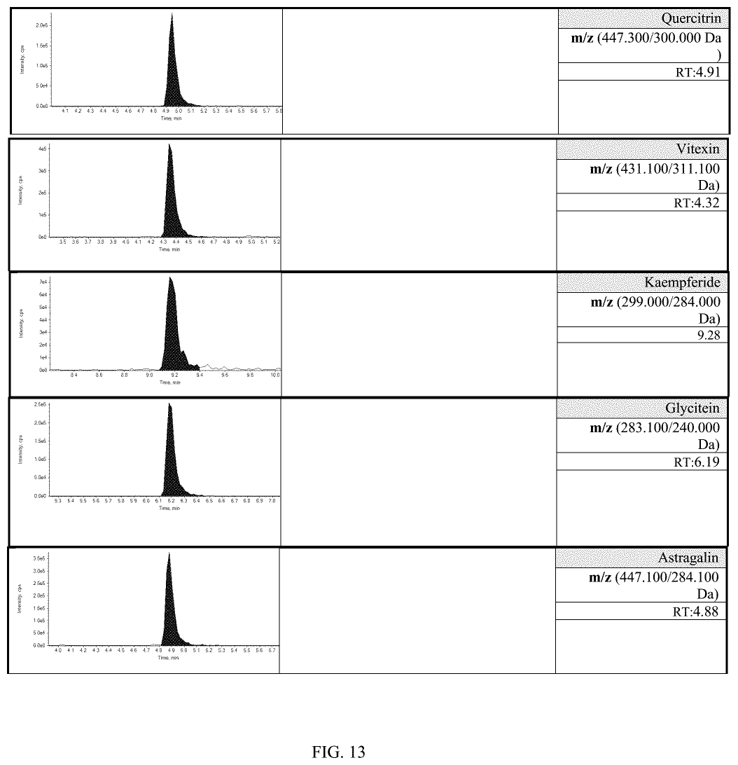

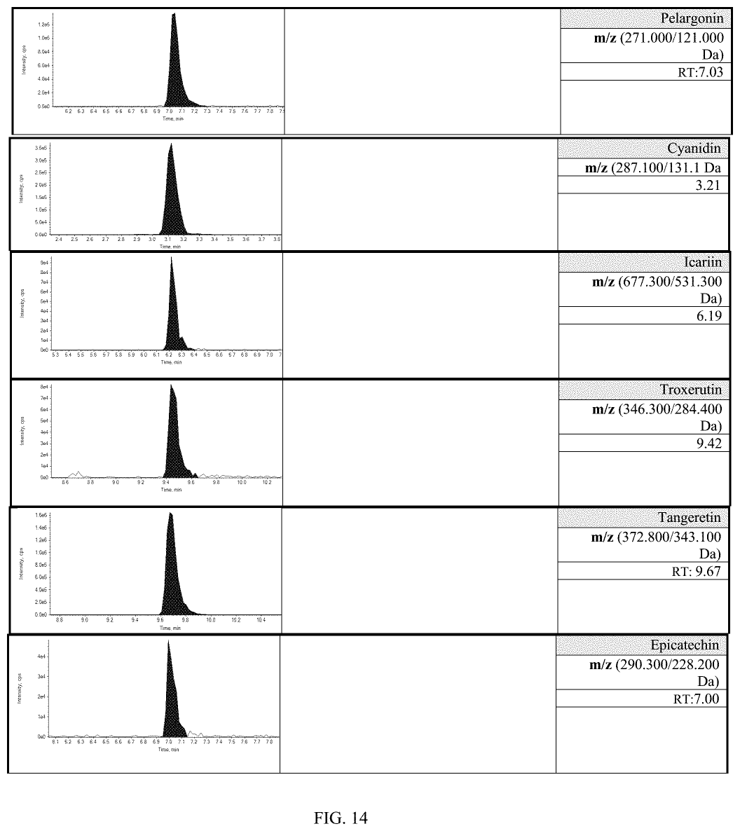

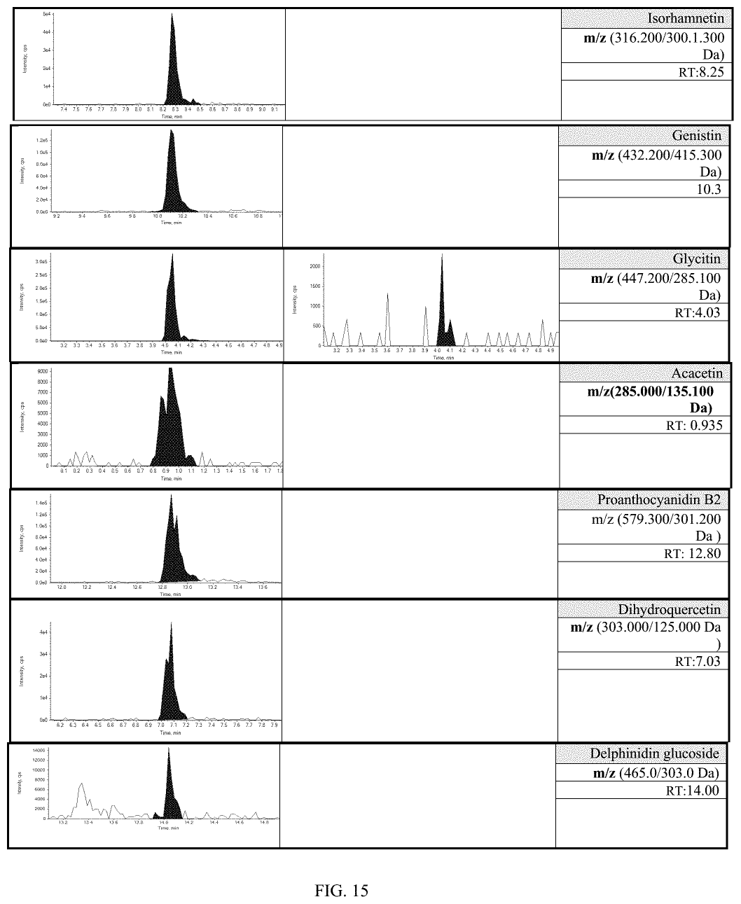

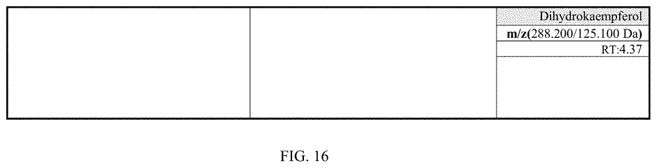

Flavonoid products are detected from the fermentation liquor of the strain CH18 based on the HPLC-MS/MS targeted metabolome, thus obtaining the types of flavonoids synthesized from the strain. The total ion current (TIC) of sample detection of the strain CH18 is shown in FIG. 1. MRM acquisition parameters and detection results of flavonoid components are shown in Table 2. The MRM chromatograms of 49 flavonoid products detected in standards and the sample of the strain CH18 are shown in FIG. 8-FIG. 16.

TABLE 2
MRM Acquisition Parameters and Detection
Results of CH18 Metabolic Flavonoids

| | | | Detected Ion Pair | | | | |
|---|---|---|---|---|---|---|---|
| | | Retention | Parent | Daughter | | | |
| Serial | | Time | Ion | Ion | | | |
| No. | Component | (min) | m/z | m/z | DP | DP | DP |
| 1 | Silybin | 6.83 | 481.2 | 124.9 | −65 | −35 | −7 |
| 2 | Puerarin | 3.24 | 415.2 | 295.1 | −56 | −31 | −16 |
| 3 | Quercetin | 5.86 | 300.8 | 271.1 | −87 | −37 | −8 |
| 4 | Genistein | 7.10 | 269.1 | 132.9 | −47 | −38 | −22 |
| 5 | Apigenin | 7.05 | 269.0 | 116.9 | −75 | −46 | −6 |
| 6 | Baicalein | 7.25 | 269.0 | 139.0 | −74 | −44 | −16 |
| 7 | Icariin | 6.19 | 677.3 | 531.3 | 13 | 20 | 8 |
| 8 | Baicalin | 16.00 | 445.1 | 268.9 | −69 | −28 | −15 |
| 9 | rutin | 4.17 | 609.1 | 300.0 | −46 | −52 | −19 |
| 10 | Hesperetin | 7.45 | 301.0 | 163.9 | −100 | −31 | −9 |
| 11 | Troxerutin | 9.42 | 743.2 | 435.0 | 27 | 32 | 17 |
| 12 | Hesperidin | 5.06 | 609.2 | 301.1 | −42 | −34 | −18 |
| 13 | (+)-catechin | 16.0 | 289.0 | 203.0 | −103 | −27 | −19 |
| 14 | Kaempferol | 16.3 | 284.5 | 93.0 | −170 | −43 | −5 |
| 15 | Naringenin | 7.08 | 270.9 | 150.9 | −77 | −25 | −8 |
| 16 | Epigallocatechin gallate | 7.04 | 457.1 | 169.0 | −92 | −24 | −10 |
| 17 | Luteolin | 6.28 | 284.7 | 132.9 | −150 | −43 | −7 |
| 18 | Myricetin | 7.17 | 317.0 | 150.9 | −80 | −32 | −8 |
| 19 | Diosmin | 4.94 | 607.1 | 299.1 | −81 | −34 | −16 |
| 20 | Dihydromyricetin | 1.86 | 319.0 | 193.1 | −115 | −16 | −6 |
| 21 | Distylin | 16.3 | 303.0 | 285.0 | −117 | −16 | −9 |
| 22 | Morin | 5.85 | 301.0 | 150.9 | −51 | −27 | −8 |
| 23 | Fisetin | 3.99 | 285.0 | 121.0 | −121 | −35 | −7 |
| 24 | Chrysin | 8.88 | 253.0 | 63.0 | −170 | −50 | −7 |
| 25 | Neohesperidin | 5.01 | 609.1 | 301.1 | −18 | −45 | −9 |
| 26 | Myricetrin | 3.26 | 463.1 | 270.9 | −101 | −54 | −15 |
| 27 | Isoliquiritigenin | 7.74 | 254.9 | 119.0 | −69 | −31 | −6 |
| 28 | Hyperoside | 4.45 | 463.1 | 300.0 | −63 | −36 | −9 |
| 29 | Tangeretin | 9.67 | 372.8 | 343.1 | 23 | 36 | 11 |
| 30 | Epicatechin | 7.00 | 290.0 | 109.0 | 60 | 28 | 13 |
| 31 | Astilbin | 8.81 | 449.2 | 97.0 | −82 | −30 | −5 |
| 32 | Phloretin | 6.98 | 272.8 | 167.0 | −93 | −22 | −5 |
| 33 | Phloretin | 3.93 | 415.0 | 253.0 | −58 | −23 | −8 |
| 34 | Isorhamnetin | 8.25 | 316.2 | 300.1 | 25 | 30 | 15 |
| 35 | Genistin | 10.3 | 432.2 | 415.3 | 38 | 11 | 13 |
| 36 | Biochanin A | 9.25 | 283.2 | 268.0 | −138 | −29 | −15 |
| 37 | Glycitin | 4.03 | 447.2 | 285.1 | 11 | 21 | 16 |
| 38 | Quercitrin | 4.91 | 447.3 | 300.0 | −89 | −35 | −9 |
| 39 | Vitexin | 4.32 | 431.1 | 311.1 | −83 | −30 | −17 |
| 40 | Kaempferide | 9.28 | 299.0 | 284.0 | −95 | −30 | −8 |
| 41 | Glycitein | 6.19 | 283.1 | 240.0 | −106 | −35 | −7 |
| 42 | Astragalin | 4.88 | 447.1 | 284.1 | −38 | −36 | −9 |
| 43 | Acacetin | 0.935 | 285.0 | 135.1 | 156 | 28 | 9 |
| 44 | Proanthocyanidin B2 | 12.80 | 579.3 | 301.2 | 30 | 17 | 6 |
| 45 | Dihydroquercetin | 7.03 | 303.0 | 125.0 | −133 | −27 | −7 |
| 46 | Cyanidin | 3.21 | 287.1 | 137.1 | −133 | −15 | −10 |
| 47 | Pelargonin | 7.03 | 271.0 | 121.0 | 139 | 43 | 7 |
| 48 | Delphinidin glucoside | 14.00 | 465.0 | 303.0 | 108 | 28 | 25 |
| 49 | Dihydrokaempferol | 4.37 | 288.2 | 125.1 | 110 | 15 | 10 |
| 50 | Quercetin D5 | 6.30 | 306.0 | 152.9 | −140 | −31 | −13 |

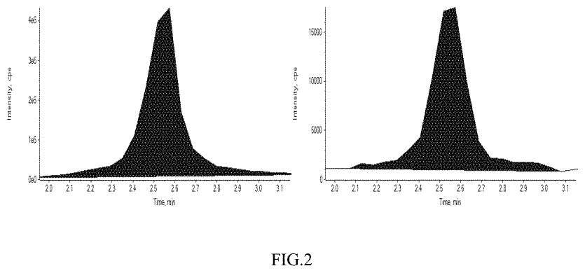

It can be seen from Table 2 that 49 flavonoids synthesized from the strain CH18 are detected from the targeted metabolome, wherein four anthocyanins exist: pelargonin, cyanidin, delphinidin glucoside and proanthocyanidin B2, which fills the gap in the study of anthocyanin components of microbial metabolism. The MRM chromatogram of delphinidin glucoside synthesized from the strain CH18 is shown in FIG. 2.

In addition to anthocyanins synthesized from the strain CH18, 45 other flavonoids synthesized from the strain CH18 are detected, which belong to flavonol, isoflavone, flavanone, flavonoid, chalcone and dihydroflavone (dihydroflavonol), wherein the flavonol refers to one or more of quercetin, rutin, myricetrin, kaempferide, troxerutin, icariin, hyperoside, fisetin, isorhamnetin, astragalin, myricetin, kaempferol and quercitrin. The isoflavone refers to one or more of puerarin, genistein, morin, daidzin, genistin, biochanin A, glycitin and glycitein. The flavanone (flavanonol) refers to one or more of (+)-catechin, epigallocatechin gallate and epicatechin. The chalcone refers to one or more of phloretin and isoliquiritigenin, and the dihydroflavone (dihydroflavonol) refers to one or more of silybin, hesperidin, distylin, neohesperidin, astilbin, naringenin, hesperetin, dihydroquercetin, dihydrokaempferol and dihydromyricetin; and the flavonoid refers to one or more of apigenin, baicalein, baicalin, luteolin, diosmin, chrysin, vitexin, acacetin and tangeretin.

In addition, naringenin, dihydrokaempferol, dihydromyricetin and dihydroquercetin detected from the HPLC-MS/MS targeted metabolome are key intermediate products in the pathway for synthesizing anthocyanins from the strain CH18, and three pathway s for synthesizing anthocyanins from strain CH18 are obtained through parsing: a dihydrokaempferol (DHK) pathway, a dihydroquercetin (DHQ) pathway and a dihydromyricetin (DHM) pathway, which can be used to regulate and simulate the production of anthocyanin compounds by fermentation of the strain CH18.

The amounts of 49 flavonoids synthesized from the strain CH18 and obtained through quantitative detection of the targeted metabolome are added to obtain the content of the flavonoids synthesized from the strain CH18. The results show that the total content of various flavonoids will change with different fermentation media and different fermentation times, and the total content is 3.38-104.80 mg/L.

In summary, the strain CH18 can be used for the production of various anthocyanins and other flavonoid components as well as the production of multiple flavonoid compounds. Various products can be used in the fields of medicines, health care, foods, cosmetics, pH color indicators, agriculture, plant protection, and antioxidants for industrial organic preparations individually or in combination.

Embodiment 3 HPLC-MS/MS Untargeted Detection of Anthocyanins Synthesized from Strain CH18

1. Method for HPLC-MS/MS Untargeted Cetection of Sample of Strain CH18

1) Instruments and Chromatographic Separation Conditions:

Instrumental analysis platform: LC-MS (Waters, UPLC; Thermo, Q Exactive), and chromatographic column (ACQUITY UPLC HSS T3 (2.1*100 mm 1.8 μm)

2) Chromatographic Separation Conditions:

Column temperature: 40° C.; sample size: 5 μL; flow rate: 0.3 mL/min; mobile phase composition: A: water+0.05% formic acid, and B: acetonitrile. The gradient elution program of the mobile phase is shown in Table 3.

TABLE 3
Gradient Elution Program of Mobile Phase

| Time (min) | A (%) | B (%) |
|---|---|---|
| 0.00 | 95 | 5 |
| 1.00 | 95 | 5 |
| 12.00 | 5 | 95 |
| 13.50 | 5 | 95 |
| 13.60 | 95 | 5 |
| 16.00 | 95 | 5 |

3) Mass Spectrometric Detection Parameters:

Positive mode: heater temperature: 300° C.; sheath gas flow rate: 45 arb; auxiliary gas flow rate: 15 arb; tail gas flow rate: 1 arb; electron spray voltage: 3.0 KV; capillary temperature: 350° C.; and S-Lens RF Level: 30%.

Negative mode: heater temperature: 300° C.; sheath gas flow rate: 45 arb; auxiliary gas flow rate: 15 arb; tail gas flow rate: 1 arb; electron spray voltage: 3.2 KV; capillary temperature: 350° C.; and S-Lens RF Level: 60%.

Scanning mode: Primary full scan (Full Scan, m/z 70-1050) and data-dependent secondary mass spectrometry scanning (dd-MS2, TopN=10); resolution: 70,000 (primary mass spectrometry) and 17,500 (secondary mass spectrometry). Collision mode: high-energy collisional dissociation (HCD).

2. Detection Results of Types of Anthocyanins Synthesized from Strain CH18

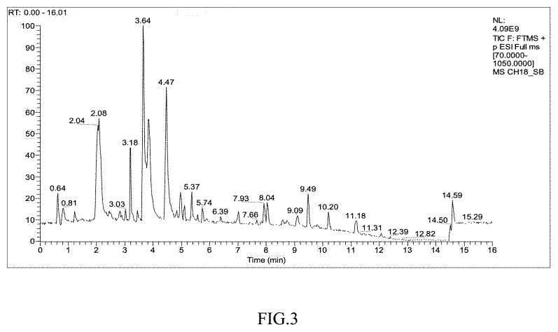

The types of anthocyanins in the fermentation sample of the strain CH18 are measured by the HPLC-MS/MS untargeted approach. The total ion current (TIC) of sample detection of the strain CH18 is shown in FIG. 3. The anthocyanin products synthesized from the strain CH18 are resolved according to the molecular weights of components obtained through liquid-mass chromatography detection and the nucleo-cytoplasmic ratio m/z of fragmentary materials to preliminarily identify six kinds of anthocyanin products: cyanindin, pelargonidin, delphinidin, peonidin, petunidin and malvidin, and the results are shown in Table 4.

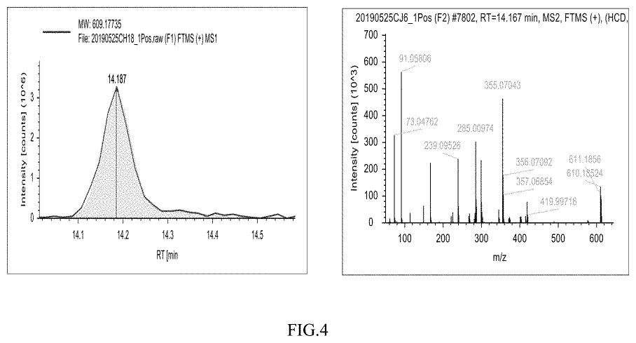

The cyanindin comprises cyanidin glucoside and cyanirin-3-O-arabinoside; the pelargonidin comprises pelargonin 3-sophorose 5-glucoside, pelargonin-3,5-glucoside and pelargonin-3-glucoside; the delphinidin comprises delphinidin-3-O-arabinoside; the peonidin comprises peonidin 3-rhamnose-5-glucoside, peonidin glucoside and peonidin-3-O-arabinoside; the petunidin comprises petunidin glucoside and petunidin-3-O-arabinoside; and the malvidin comprises malvidin-3-O-glucoside. The measured mass spectrogram of peonidin 3-rhamnose-5-glucoside as an example is shown in FIG. 4.

TABLE 4
Detection Results of Anthocyanins

| | | | Molecule, Ion | |
|---|---|---|---|---|
| | Retention | | (M)+ or | |
| Serial | Time | Ionization | (M − H)⁻ | Preliminarily Identified |
| No. | (min) | Mode | (m/z) | Component |
| 1 | 8.099 | Negative | 498.0111 | Peonidin glucoside |
| | | mode | | |
| 2 | 1.803 | Negative | 468.1295 | Peonidin-3-O-arabinoside |
| | | mode | | |
| 3 | 14.167 | Positive mode | 609.1774 | Peonidin 3-rhamnose-5- |
| | | | | glucoside |
| 4 | 3.866 | Negative | 470.1267 | Delphinidin-3-O- |
| | | mode | | arabinoside; |
| 5 | 3.592 | Negative | 449.1435 | Cyanidin glucoside |
| | | mode | | |
| 6 | 14.632 | Negative | 419.7777 | Cyanirin-3-O-arabinoside |
| | | mode | | |
| 7 | 2.882 | Positive mode | 352.1414 | Petunidin |
| 8 | 10.401 | Negative | 479.3011 | Petunidin glucoside |
| | | mode | | |
| 9 | 12.005 | Positive mode | 484.2818 | Petunidin-3-O-arabinoside |
| 10 | 3.86 | Negative | 630.1538 | Pelargonin-3,5-glucoside |
| | | mode | | |
| 11 | 12.952 | Positive mode | 757.2147 | Pelargonin 3-sophorose 5- |
| | | | | glucoside |
| 12 | 9.049 | Negative | 468.308 | Pelargonin-3-glucoside |
| | | mode | | |
| 13 | 9.661 | Negative | 493.2500 | Malvidin-3-O-glucoside |
| | | mode | | |
| 14 | 10.628 | Positive mode | 576.1256 | Proanthocyanidin A2 |

Embodiment 4 Coloration and Anti-Oxidation of Flavonoids such as Anthocyanins from Strain CH18

1. Anthocyanin Pigment Substance from Strain CH18

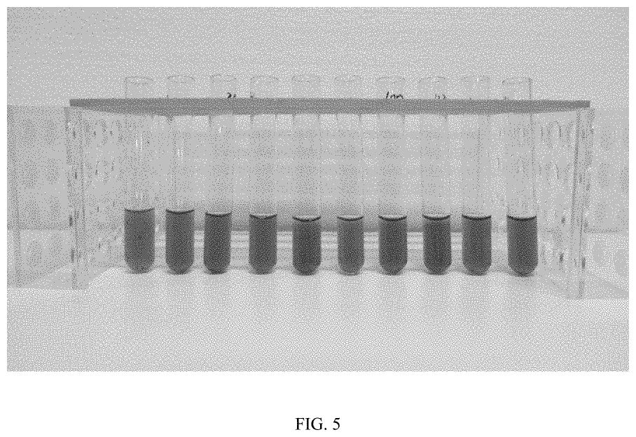

The pH value of the fermentation liquor of the strain CH18 is respectively regulated to 2, 3, 4, 5, 6, 7, 8, 9, 10 and 11, and the fermentation liquor presents different colors, as shown in FIG. 5, and turns red in acidic conditions and blue to blue grey in alkaline conditions. The anthocyanins of the strain CH18, as a coloration substance, can be used in the fields of water quality determination, body fluid determination, acid-base indicators, foods, health care products, cosmetics and agriculture.

In the aspect of pigments, with the mass use of artificial synthetic pigments, a series of environmental and health problems have been caused, which increase the demand of people for natural pigments. Natural pigments are mainly derived from plants and microorganisms. Plants have a long growth cycle and thus are limited in large-scale applications. Compared with plant-derived natural pigment, microorganism-derive pigments, such as anthocyanin pigments from CH18, are easy for large-scale rapid fermentation and production, and has a broad application prospect.

2. Determination of Anti-Oxidation and Stability of Flavonoids from Strain CH18

1) Anti-Oxidation of Flavonoids from Strain CH18

Determination of Scavenging Effect of Flavonoids from Strain CH18 on DPPH Free Radicals:

2 mL of fermentation liquor of the strain CH18 is drawn into a test tube, added with 2 mL of 0.25 mmol/L 1,1-diphenyl-2-trinitrophenylhydrazine (DPPH) ethanol solution, and mixed well. The mixed solution is kept standing at room temperature for 30 min, and absorbance is measured at the wave length of 517 nm and recorded as A1. 2 mL of fermentation extracting solution of the strain CH18 is mixed with 2 mL of absolute ethyl alcohol uniformly, and absorbance is measured and recorded as A2; and 2 mL of DPPH ethanol solution is mixed with 2 mL of absolute ethyl alcohol solution uniformly, and absorbance is measured and recorded as A3. Vitamin C (Vc) is used as positive control. The DPPH free radical scavenging rate of the flavonoids synthesized from the strain CH18 on is calculated according to the formula below:

DPPH scavenging rate (%)=[1-(A1−A2)÷A3]×100

Researches show that with the VC addition amount of 0.08 mg/ml and 0.18 mg/ml, the DPPH scavenging rate of the flavonoids in the fermentation extracting solution of the strain CH18 is 7.1% and 5.5% higher than that of Vc solution, respectively. The extracting solution of the strain CH18 has high scavenging capacity and antioxidant activity on DPPH free radicals, and can be used in the fields of foods, medicines, health care products, cosmetics, agriculture and industry and as antioxidant.

2) Stability of Anthocyanins from Strain CH18

a) Impacts of Light and Dark on Stability

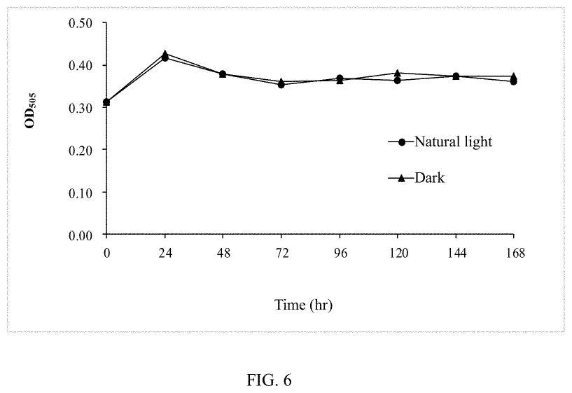

The impacts of light and dark on the stability of anthocyanins from the strain CH18 are compared at room temperature (20-37° C.). According to the characteristic that the anthocyanins synthesized from the strain CH18 have an absorption peak at 505 nm, the variation value of the anthocyanin solution OD₅₀₅ of the strain CH18 is measured to determine the stability. The determination results are shown in FIG. 6. Researches show that the anthocyanins from the strain CH18 have good stability under light and dark conditions, and can be applied and preserved under natural light and dark conditions.

b) Impacts of Metal Ions on Stability

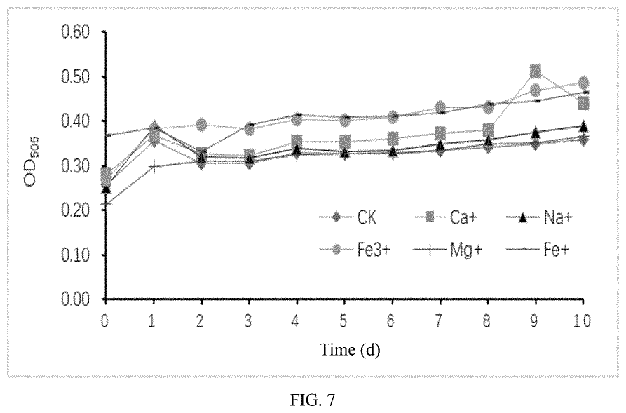

Ca²⁺, Na⁺, K⁺, Mg²⁺, Fe⁺ and Fe³⁺ salt ions are added into the anthocyanin extracting solution of the strain CH18 and put in a 5° C. refrigerator, and the OD₅₀₅ value and the color change of the solution are observed. The determination results are shown in FIG. 7. The results show that the OD₅₀₅ value of the solution of the experimental group added with Ca²⁺, Na⁺, Mg²⁺, Fe⁺ and Fe³⁺ metal ions is equal to or high than that of the control group not added with salt ions, which indicates that the addition of the above metal ions will not affect the stability of anthocyanins from CH18 and the addition of some metal ions also can improve the stability of anthocyanins from CH18. Therefore, the anthocyanins from strain CH18 have good stability when being added to foods, health care products, medicines, water bodies, cosmetics and other products containing the above metals.

Embodiment 5 Application of Strain CH18 in Industry

1. Foods, Medicines and Health Care Products

1) Application in food field: fermentation liquor and solid fermentation product are obtained by liquid fermentation of the strain CH18 or solid fermentation of raw materials such as rice and other auxiliary materials such as bran as a medium, and the mixture or simple components extracted from the fermentation liquor and solid fermentation product or the fermentation liquor and solid fermentation product directly used as the raw material of anthocyanin are added or applied to jam, jelly, acid beverage or alcoholic beverages, and can also be used in the food industry of dairy products, meat products, wine processed products, flour products, fruits, vegetables or pickles. Because of strong anti-oxidation, stability and biological activity, the strain CH18 is helpful to prolong the preservation of foods and improve the nutritional value.

2) Application in Fields of Health Care Products and Medicines

The anthocyanins and other flavonoid products synthesized from the strain CH18 have high anti-oxidation, stability and biological activity, and can be applied in the production of health care products, medicines and additives.

2. Application in Acid-Base Indicators, Natural Pigment and Antioxidant

The anthocyanins can keep bright red, purple or blue within a wide range of pH values, have good thermal stability under acidic conditions, and can be used in foods, health care products, cosmetics, water quality testing, daily necessities, agriculture and chemical industry as natural pigment, developer, acid-base indicator and antioxidant due to strong antioxidant activity.

3. Biological Agents

According to the products synthesized from the strain CH18, antioxidant biological agents composed of multiple components can be produced, and pure anthocyanins and other flavonoids can also be obtained through separation and purification methods and used as raw materials or additives for biological agents, natural pigments, standards of biochemical components, food additives, health care capsules, powders, tablets or liquid products.

The above description of the disclosed embodiments enables those skilled in the art to realize or use the present invention. Many modifications to the above embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. Application of a strain CH18 in preparation of anthocyanins and other flavonoid compounds the strain CH18 is Fusarium oxysporum; the strain is preserved at China General Microbiological Culture Collection Center (CGMCC) on Oct. 11, 2017, the preservation address is: Building 3, Courtyard 1, Beichen West Road, Chaoyang District, Beijing, and the preservation number is CGMCC NO.14784.

2. Application of a strain CH18 in preparation of anthocyanins and other flavonoid compound products;
the preservation number of the strain CH18 is CGMCC NO.14784.

3. Application of a strain CH18 in preparation of products for resisting bacteria and virus, increasing leucocytes, resisting tumors, reducing blood pressure, blood lipid and natural pigment, scavenging free radicals or exciting the central nervous system, wherein the preservation number of the strain CH18 is CGMCC NO.14784, and bioactive substances of the products are anthocyanins and other flavonoid compounds.

4. The application according to claim 1, wherein the anthocyanins are anthocyanins or proanthocyanidins;
preferably, the proanthocyanidins comprise proanthocyanidins A and proanthocyanidins B; the proanthocyanidins A are proanthocyanidins A2; and preferably, the proanthocyanidins B are proanthocyanidins B2.

5. The application according to claim 2, wherein the anthocyanins are anthocyanins or proanthocyanidins;
preferably, the proanthocyanidins comprise proanthocyanidins A and proanthocyanidins B; the proanthocyanidins A are proanthocyanidins A2; and preferably, the proanthocyanidins B are proanthocyanidins B2.

6. The application according to claim 3, wherein the anthocyanins are anthocyanins or proanthocyanidins;
preferably, the proanthocyanidins comprise proanthocyanidins A and proanthocyanidins B; the proanthocyanidins A are proanthocyanidins A2; and preferably, the proanthocyanidins B are proanthocyanidins B2.

7. The application according to claim 4, wherein the anthocyanins comprise one or more of cyanindin, pelargonidin, delphinidin, peonidin, petunidin and malvidin. preferably, the cyanindin comprises one or more of cyanidin, cyanidin glucoside and cyanirin-3-O-arabinoside;
preferably, the pelargonidin comprises one or more of pelargonin, pelargonin 3-sophorose 5-glucoside, pelargonin-3,5-glucoside and pelargonin-3-glucoside;
preferably, the delphinidin comprises one or more of delphinidin glucoside and delphinidin-3-O-arabinoside;
preferably, the peonidin comprises one or more of peonidin 3-rhamnose-5-glucoside, peonidin glucoside and peonidin-3-O-arabinoside;
preferably, the petunidin comprises petunidin glucoside or petunidin-3-O-arabinoside;
preferably, the malvidin refers to malvidin-3-O-glucoside.

8. The application according to claim 5, wherein the anthocyanins comprise one or more of cyanindin, pelargonidin, delphinidin, peonidin, petunidin and malvidin. preferably, the cyanindin comprises one or more of cyanidin, cyanidin glucoside and cyanirin-3-O-arabinoside;
preferably, the pelargonidin comprises one or more of pelargonin, pelargonin 3-sophorose 5-glucoside, pelargonin-3,5-glucoside and pelargonin-3-glucoside;
preferably, the delphinidin comprises one or more of delphinidin glucoside and delphinidin-3-O-arabinoside;
preferably, the peonidin comprises one or more of peonidin 3-rhamnose-5-glucoside, peonidin glucoside and peonidin-3-O-arabinoside;
preferably, the petunidin comprises petunidin glucoside or petunidin-3-O-arabinoside;
preferably, the malvidin refers to malvidin-3-O-glucoside.

9. The application according to claim 6, wherein the anthocyanins comprise one or more of cyanindin, pelargonidin, delphinidin, peonidin, petunidin and malvidin. preferably, the cyanindin comprises one or more of cyanidin, cyanidin glucoside and cyanirin-3-O-arabinoside;
preferably, the pelargonidin comprises one or more of pelargonin, pelargonin 3-sophorose 5-glucoside, pelargonin-3,5-glucoside and pelargonin-3-glucoside;
preferably, the delphinidin comprises one or more of delphinidin glucoside and delphinidin-3-O-arabinoside;
preferably, the peonidin comprises one or more of peonidin 3-rhamnose-5-glucoside, peonidin glucoside and peonidin-3-O-arabinoside;
preferably, the petunidin comprises petunidin glucoside or petunidin-3-O-arabinoside;
preferably, the malvidin refers to malvidin-3-O-glucoside.

10. The application according to claim 1, wherein the other flavonoid compounds belong to one or more of flavonol, isoflavone, flavanone, flavonoid, chalcone and dihydroflavone (dihydroflavonol);
preferably, the flavonol comprises one or more of quercetin, rutin, myricetrin, kaempferide, troxerutin, icariin, hyperoside, fisetin, isorhamnetin, astragalin, myricetin, kaempferol and quercitrin. preferably, the isoflavone comprises one or more of puerarin, genistein, morin, daidzin, genistin, biochanin A, glycitin and glycitein;
preferably, the flavanone (flavanonol) comprises one or more of (+)-catechin, epigallocatechin gallate and epicatechin;
preferably, the chalcone comprises one to more of phloretin and isoliquiritigenin;
preferably, the dihydroflavone (dihydroflavonol) comprises one or more of silybin, hesperidin, distylin, neohesperidin, astilbin, naringenin, hesperetin, dihydroquercetin, dihydrokaempferol and dihydromyricetin;
preferably, the flavonoid comprises one or more of apigenin, baicalein, baicalin, luteolin, diosmin, chrysin, vitexin, acacetin and tangeretin.

11. The application according to claim 2, wherein the other flavonoid compounds belong to one or more of flavonol, isoflavone, flavanone, flavonoid, chalcone and dihydroflavone (dihydroflavonol);
preferably, the flavonol comprises one or more of quercetin, rutin, myricetrin, kaempferide, troxerutin, icariin, hyperoside, fisetin, isorhamnetin, astragalin, myricetin, kaempferol and quercitrin. preferably, the isoflavone comprises one or more of puerarin, genistein, morin, daidzin, genistin, biochanin A, glycitin and glycitein;
preferably, the flavanone (flavanonol) comprises one or more of (+)-catechin, epigallocatechin gallate and epicatechin;
preferably, the chalcone comprises one to more of phloretin and isoliquiritigenin;
preferably, the dihydroflavone (dihydroflavonol) comprises one or more of silybin, hesperidin, distylin, neohesperidin, astilbin, naringenin, hesperetin, dihydroquercetin, dihydrokaempferol and dihydromyricetin;
preferably, the flavonoid comprises one or more of apigenin, baicalein, baicalin, luteolin, diosmin, chrysin, vitexin, acacetin and tangeretin.

12. The application according to claim 3, wherein the other flavonoid compounds belong to one or more of flavonol, isoflavone, flavanone, flavonoid, chalcone and dihydroflavone (dihydroflavonol);
preferably, the flavonol comprises one or more of quercetin, rutin, myricetrin, kaempferide, troxerutin, icariin, hyperoside, fisetin, isorhamnetin, astragalin, myricetin, kaempferol and quercitrin. preferably, the isoflavone comprises one or more of puerarin, genistein, morn, daidzin, genistin, biochanin A, glycitin and glycitein;
preferably, the flavanone (flavanonol) comprises one or more of (+)-catechin, epigallocatechin gallate and epicatechin;
preferably, the chalcone comprises one to more of phloretin and isoliquiritigenin;
preferably, the dihydroflavone (dihydroflavonol) comprises one or more of silybin, hesperidin, distylin, neohesperidin, astilbin, naringenin, hesperetin, dihydroquercetin, dihydrokaempferol and dihydromyricetin;
preferably, the flavonoid comprises one or more of apigenin, baicalein, baicalin, luteolin, diosmin, chrysin, vitexin, acacetin and tangeretin.

13. The application according to claim 3, wherein the products are foods, health care products, medicines, cosmetics and agricultural or daily chemical products.

14. The application according to claim 13, wherein the foods are dairy products, meat products, wine processed products, alcoholic beverages, acid beverage, flour products or pickles.

15. The application according to claim 13, wherein the health care products, medicines, cosmetics or natural pigments are bacteria powder, tablets, capsules, paste or liquid.

16. The application according to claim 13, wherein the agricultural or daily chemical products are pH test liquid, test powder, test paper and additives.
